(19) United States
(12) Patent Application Publication
(10) Pub. No.: US 2009/0099419 A1
(43) Pub. Date: Apr. 16, 2009

(54) SWITCHING MECHANISM FOR SWALLOWABLE MEDICAL DEVICE

(75) Inventor: Kentaro MATSUMOTO, Saitama (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/247,450

(22) Filed: Oct. 8, 2008

(30) Foreign Application Priority Data
Oct. 12, 2007 (JP) 2007-266816

(51) Int. Cl. A61B 1/00

(52) U.S. Cl. 600/118

(57) ABSTRACT

A switching mechanism for a swallowable medical device has first and second contacts and an expandable member. The medical device has a shell and a first circuit unit provided in the shell. The first and second contacts are electrically connectable with each other, and an operation of the first circuit unit is switched by electrically connecting the first and second contact. The expandable member is capable of expanding by absorbing a predetermined liquid. The expandable member displaces the second contact by expanding such that the second contact gets electrically connected to or disconnected from the first contact.

### BACKGROUND OF THE INVENTION

1. Field of the Invention

The present invention relates to a switching mechanism for a swallowable medical device such as a capsule endoscope, and in particular, for switching an operation of the medical device when initiating use.

2. Description of the Related Art

A capsule endoscope is well known as an endoscope for observing the insides of a human body such as the stomach or intestines. Circuits in the capsule endoscope are driven by a power-supply unit such as a battery which is provided in the capsule endoscope, but they can be driven only for a short period by the battery because of the limited available charge. Therefore, the capsule endoscope is usually turned on by the doctor just before being swallowed by the patient, in order to reduce unnecessary power consumption. Furthermore, it is preferable that the endoscope be turned on by a simple operation and without disassembling in order to ensure a seal of the interior.

Conventionally, a switching mechanism utilizing a magnet is known as a means for turning on the power supply of the capsule endoscope, as shown in International Publication NO. WO/2001/035813. In this mechanism, the capsule endoscope is stored in a case with a magnet, and when the capsule endoscope is removed from the case the capsule endoscope is turned on automatically by the change in magnetic field. This mechanism does not require the disassembly of the capsule endoscope and can provide an easy power-on operation.

However, in this mechanism, all the cases must have a magnet, and a number of magnets corresponding to cases or endoscopes are necessary. In addition, the case and the in endoscope are usually single-use. Accordingly, a large number of magnets are required. Furthermore, in this mechanism, the power supply can be started by removing the capsule endoscope from the case. However, after swallowing, the long time it takes for the endoscope to reach the region to be observed by a doctor (such as the intestines or the stomach), wastes valuable electrical power. This requires a larger battery, increasing the size and manufacturing cost of the capsule endoscope.

In addition, when the capsule endoscope is powered on, it emits radio waves in order to transmit data such as image data. While the endoscope is still outside the body, the unmitigated radio waves may adversely affect peripheral devices.

### SUMMARY OF THE INVENTION

Therefore, an object of the present invention is to provide a simple switching mechanism for a swallowable medical device, which can alter the operation of a circuit after the device is swallowed, without the use of a magnet.

According to the present invention, there is provided a switching mechanism for a swallowable medical device having a shell and a first circuit unit provided in an interior of the shell. The switching mechanism has first and second contacts and an expandable member. The first and second contacts are electrically connectable with each other and provided in the interior. The first circuit unit is operated by electrically connecting the first and second contacts. The expandable member is capable of expanding by absorbing a predetermined liquid. The expandable member expands and thereby displaces the second contact such that the second contact gets electrically connected to or disconnected from the first contact.

### BRIEF DESCRIPTION OF THE DRAWINGS

The objects and advantages of the present invention will be better understood from the following description, with reference to the accompanying drawings in which:

FIG. 1 is a schematic view, partly sectioned, of a capsule endoscope in the first embodiment of the present invention;

FIG. 2 is a schematic view, partly sectioned, of a capsule endoscope in the second embodiment of the present invention; and

FIG. 3 is a schematic view, partly sectioned, of a capsule endoscope in the third embodiment of the present invention.

### DESCRIPTION OF THE PREFERRED EMBODIMENTS

The present invention will be described below with reference to the embodiments shown in the drawings.

A switching mechanism for the capsule endoscope is explained below, but the switching mechanism in these embodiments can be applied to any other swallowable medical devices.

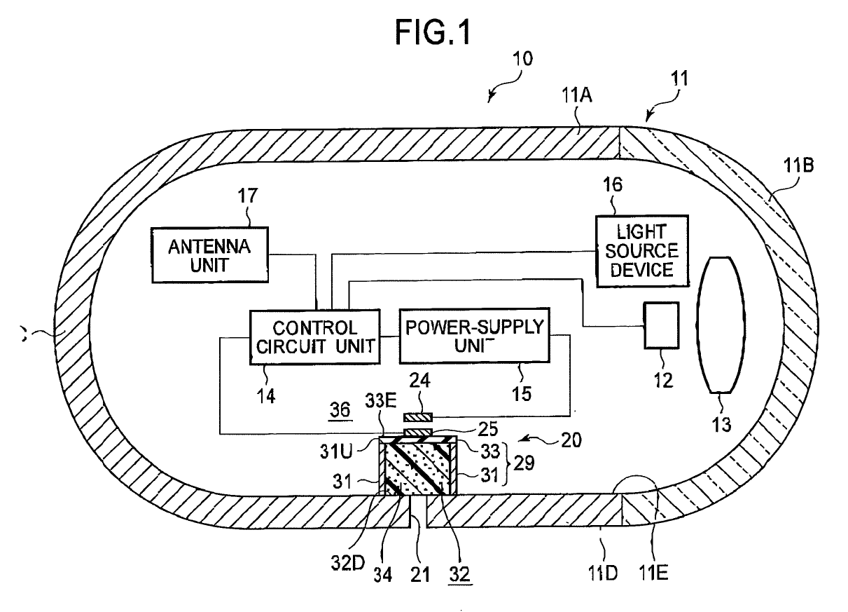

FIG. 1 shows a capsule endoscope in the initial state before the power supply is turned on in the first embodiment. The capsule endoscope 10 is a swallowable medical device for observing the inside of a human body and enters the human body by being swallowed. The capsule endoscope 10 has a shell 11 which seals its interior. The shell 11 has a body 11A which is opaque and cylindrical, a transparent cover 11B which covers one end of the body 11A, and an opaque cover 11C which covers the other end thereof. The covers 11B and 11C are dome-shaped. In FIG. 1, the main axis of the body 11A extends in the horizontal direction.

The capsule endoscope 10 has an imaging device 12, a lens system 13, a control circuit unit (a first circuit unit) 14, a power-supply unit (a second circuit unit) 15 such as a battery, a light source device 16, and an antenna unit 17, all inside the space of the shell 11. The power-supply unit 15 supplies the electrical power to the control circuit unit 14, and the control circuit unit 14 operates with the supplied electrical power. The control circuit unit 14 has several circuits including a driving circuit for driving the devices 12 and 16 and the antenna unit 17.

The lens system 13 forms an object image which is illuminated by the light emitted from the light source device 16 on the imaging device 12. The imaging device 12 captures the object image and generates the image data from the object image. The image data is transmitted to the outside of the capsule endoscope 10 by the antenna unit 17 through radio waves. The radio waves are received by a data-receiving apparatus which is located outside of the capsule endoscope 10.

The capsule endoscope 10 has a switching mechanism 20. The switching mechanism 20 has first and second contacts 24 and 25, a partition member (first cover member) 29, and an expandable member 32. In this embodiment, the switching mechanism 20 is used for turning on the power supply of the control circuit unit 14 (namely, the endoscope 10) by connecting the first and second contacts. The switching mechanism may also be used for altering any kind of operation of the control circuit unit 14.

The partition member 29 has a side wall 31 and an elastic cover member 33 which is an elastic membrane. The side wall 31 which surrounds a part of the interior of the shell 11 projects inward from an inner surface 11E of the body 11A. The part of the interior of the shell 11 which is surrounded by the side wall 31 is defined as defined space 32.

The elastic cover member 33 which covers the defined space 32 and the periphery 33E of the elastic cover member 33 is hermetically bonded to a top end 31U. The top of the defined space 32 is sealed by the elastic cover member 33. Accordingly, the partition member 29 which separates the defined space 32 from the remaining space 36 in the interior of the shall 11 prevents water or liquid from leaking from the defined space 32 to the space 36. Namely, the space 36 is a sealed from its surroundings including the defined space 32.

The shell 11 has a hole 21 which penetrates the body 11A. The hole 21 is located at a part of the body 11A that is composed of the bottom 32D of the defined space 32. The defined space 32 communicates with the outside of the shell 11 through the hole 21. Liquid can enter the defined space 32 through the hole 21 without entering the space 36, because the defined space 32 is sealed from the space 36 by the partition member 29. Namely, the hole 21 is covered and sealed by the partition member 29 on the inner side of the shell 11.

The elastic cover member 33 is formed of an elastic material such as rubber or resin and the like. Any kind of rubber or resin may be used as the elastic material. For example, the elastic cover member 33 may be formed of a biocompatible rubber. The elastic cover member 33 is preferably formed of an insulating material so as to isolate itself from the second contact 25.

The expandable member 34 is arranged in the defined space 32. Namely, the expandable member 34 is arranged in a space between the partition member 29 and the inner surface 11E of the shell 11. The expandable member 34 is formed of a fibrous material or a sponge, for example. The expandable member 34 is capable of expanding by absorbing liquid (e.g. aqueous liquid) such as water or bodily fluid. The bodily fluid may be a digestive fluid, for example. Examples of digestive fluids include saliva, gastric fluid, intestinal fluid, and bile.

In the shell 11, the first and second contacts 24 and 25 which are made of a conductive material such as metal are arranged. The second contact 25 is fixed on the top surface (inner surface) of the elastic cover member 33. The first contact 24 is located on the inner side of the second contact 25 and the first and second contacts 24 and 25 face each other. The first contact 24 is mounted to the shell 11 through a holder (not shown in FIG. 1).

The power-supply unit 15 and the control circuit unit 14 electrically connect with the first and second contacts 24 and 25, respectively, via electric leads and the like. When the second contact 25 contacts with the contact 24, the electrical power is supplied from the power-supply unit 15 to the control circuit unit 14. The control circuit unit 14 to which the electrical power is supplied operates so as to drive the light source device 16, the imaging device 12, and the antenna, unit 17 as described above.

While the second contact 25 electrically disconnects from the first contact 24 by separating from the contact 24, electrical power is no longer supplied to the control circuit unit 14. In that case, the operation of the control circuit unit 14 is not performed and the light source device 16, the imaging device 12, and the antenna unit 17 are not driven. In the initial state, namely, before the endoscope 10 is swallowed, the expandable member 34 is dry and unexpended. Therefore, the second contact 25 is separated from the first contact 24.

When the capsule endoscope 10 is swallowed and enters the human body, the expandable member 34 absorbs digestive fluid (such as saliva) which enters the defined space 32 through the hole 21. Accordingly, the expandable member 34 is expanded. The expanding expandable member 34 urges the elastic cover member 33 inward such that the elastic cover member 33 is deformed and expanded inward while maintaining its seal with the side wall 31. As the elastic cover member 33 expands, the second contact 25 is displaced inward and then forme contact with the first contact 24. When the second contact 25 forms contact with the first contact 24, the operation of the control circuit unit 14 starts with the supply of the electrical power from the power-supply unit 15 to the control circuit unit 14.

As described above, in this embodiment, before swallowing the capsule endoscope 10, the devices 12 and 16 and the antenna unit 17 are not driven. This can reduce the power consumption prior to swallowing. Also, the antenna unit 17 starts transmitting radio waves after the endoscope 10 enters the human body. This delay prevents the radio waves from adversely affecting peripheral devices located outside of the endoscope 10, because the radio waves are partially shielded by the human body.

Furthermore, in this embodiment, the switching mechanism 20 does not require any specific operations of the user for turning the power supply on, because the operation of the control circuit unit 14 starts automatically through contact with the bodily fluid. In addition, because the power-supply unit 15 is electrically separated from the control circuit unit 14 in the initial state, the electrical power consumption in the power-supply unit 15 is more effectively reduced.

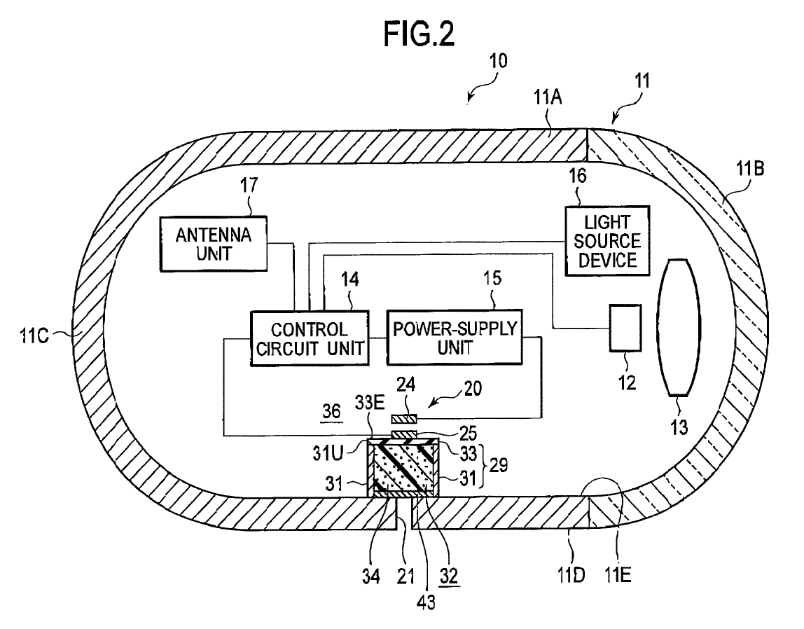

FIG. 2 shows the endoscope 10 of the second embodiment. The difference between this embodiment and the first embodiment is that the switching mechanism has at second cover member 43. The difference is explained below.

As shown in FIG. 2, the second cover member 43 which is a membrane is located on the inner surface 11E so as to cover the hole 21. The periphery of the second cover member 43 around the hole 21 is bonded to the inner surface 11E. Therefore, the second cover member 43 covers the hole 21 so as to seal the hole 21. The expandable member 34 is located on the second cover member 43. The second cover member 43 is dissolvable by a specific liquid (an aqueous liquid). The specific liquid is bodily fluid, for example, and the example of the bodily fluid is the same as that described above.

For example, the second cover member 43 is formed of a material dissolvable by a solution with a pH less than a predetermined value but not by a solution with a pH more than or equal to the predetermined value. Specifically, the second cover member 43 may be dissolved by an acidic solution but not by an alkaline or neutral solution. Therefore, even when the capsule endoscope 10 is exposed to alkaline or neutral liquid such as a bodily fluid, the liquid does not enter the defined space 32 through the hole 21 due to the obstruction of the second cover member 43 which has not dissolved.

In this embodiment, when the capsule endoscope 10 is placed in the mouth and is exposed to saliva, the second cover member 43 is not dissolved by saliva, thereby preventing the expandable member 34 from expanding. When the capsule endoscope 10 reaches the stomach, the second cover member 43 is dissolved by an acidic gastric fluid. After the second cover member 43 dissolves, the expandable member 34 absorbs the gastric fluid which enters the defined space 32 through the hole 21, and thereby expands. With the expansion of the expandable member 34, the operation of the control circuit unit 14 starts, similarly to the first embodiment.

Furthermore, the second cover member 43 may be formed of a material dissolvable by a solution with a pH more than a predetermined value but not dissolvable by a solution with a pH less than or equal to the predetermined value. In such a case, the second cover member 43 is not dissolved by saliva or acidic gastric fluid, but is dissolved in the intestines by intestinal fluid or bile which is alkaline. Accordingly, in this case, when the capsule endoscope 10 reaches the intestines, the expandable member 34 expands and the operation of the control circuit unit 14 starts.

Namely, it is preferable that the second cover member 43 be dissolvable by liquid with a pH within a predetermined range but not by liquid with a pH beyond the predetermined range. In addition, the second cover member 43 may be located on the outer surface 11D of the shell 11 or in an inner space of the hole 21 instead of on the inner surface 11E.

In this embodiment, the operation of the control circuit unit 14 starts when the endoscope 10 reaches the desired organ to be observed such as stomach or intestines. Therefore, the electrical power consumption of the power-supply unit 15 can more effectively be reduced.

In the second embodiments, the whole of the second cover member 43 is usually dissolved, but only a part of the second cover member 43 may be dissolved by the specific liquid. Therefore, the second cover member 43 may include a material which is not dissolvable by the specific liquid, but the main compound of the second cover member 43 is preferably a material dissolvable by the specific liquid.

In the second embodiment, a whole or a part of the second cover member 43 may be decomposable by the specific liquid instead of dissolvable by the specific liquid. Namely, the second cover member 43 is not limited to the above-mentioned structure, if the second cover member 43 is capable of disintegrating by contact with the specific liquid and can allow the liquid to enter the specific space 32 through the hole 21 after disintegrating.

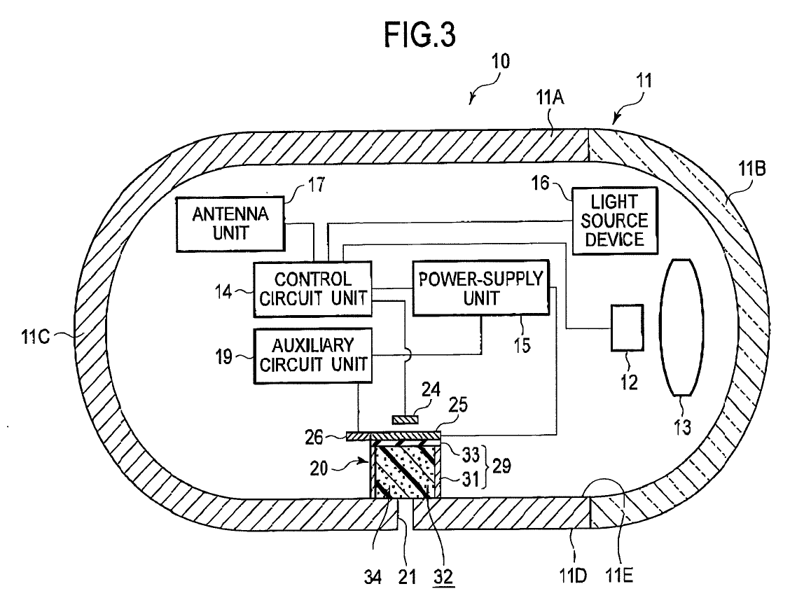

FIG. 3 shows the capsule endoscope 10 in the third embodiment. In this embodiment, the capsule endoscope 10 further has an auxiliary circuit unit 19 and a third contact 26 in the shell 11.

The auxiliary circuit unit 19 maybe a timer, for example. The auxiliary circuit unit 19 measures time using the electrical power from the power-supply unit 15. The control circuit unit 14, the power-supply unit 15, and the auxiliary circuit unit 19 electrically connect with the first, second, and third contacts 24, 25, and 26, respectively, through electric leads, for example.

In the initial state, namely, while the expandable member 34 is not expanded, the second contact 25 (e.g. the side edge of the second contact) contacts the third contact 26. Therefore, in the initial state, the auxiliary circuit unit 19 electrically connects the power-supply circuit unit 15, and the auxiliary circuit unit 19 operates with the supplied electrical power. Namely, in this embodiment, before operation of the control circuit unit 14, the waiting time is measured by the auxiliary circuit unit 19.

When the endoscope 10 is swallowed, the expandable member 34 is expanded by the predetermined liquid and thereby displaces the second contact 25, similarly to the first and second embodiments. The displaced second contact 25 is separated from the third contact 26 and makes contact with the first contact 24. Due to this, the control circuit unit 14 electrically connects to the power-supply unit 15 and operates with the power supply from the power-supply unit 15. Furthermore, the second contact 25 gets disconnected from the third contact 26. Hence, the electrical power supplied to the auxiliary circuit unit 19 stops and the timing by the auxiliary circuit unit 19 stops.

As described above, in this embodiment, the circuit unit which receives power may be switched, from the auxiliary circuit unit 19 to the control circuit unit 14 by the expansion of the expandable member 34.

In the above embodiments, the shell 11 has only one hole 21, but the shell 11 may have a plurality of fine holes for connecting the defined space 32 with the outside of the shell 11. In such a case, the amount of liquid which passes through the holes can be adjusted by changing the number of the holes. Therefore, the period from the swallow to the activation can be determined according to the user's wishes. The period from the swallow to the activation can also be determined by changing the diameter of the hole(s).

Furthermore, in the above embodiments, the inner space of the hole 21 may be defined as the defined space 32 in which the expandable member 34 is located. In such a case, the expandable member 34 may be expanded by the predetermined liquid which enters the hole 21.

In the above embodiments, the partition member 29 may not comprise an elastic member and a part of the partition member 29 may move so as to displace the second contact 25 by the expansion of the expandable member 32 instead of deform.

Although the embodiments of the present invention have been described herein with reference to the accompanying drawings, obviously many modifications and changes can be made by those skilled in this art without departing from the scope of the invention.

The present disclosure relates to subject matter contained in Japanese Patent Application No. 2007-266816 (filed on Oct. 12, 2007) which is expressly incorporated herein, by reference, in its entirety.

1. A switching mechanism for a swallowable medical device, said device comprising a shell and a first circuit unit provided in the interior of said shell, said switching mechanism comprising:
first and second contacts that are electrically connectable with each other and provided in said interior, said first circuit unit being operated when said first and second contacts are electrically connected; and
an expandable member that is capable of expanding by absorbing a predetermined liquid;
said expandable member expanding and thereby displacing said second contact such that said second contact gets electrically connected to or disconnected from said first contact.

2. The switching mechanism as claimed in claim 1, wherein said shell comprises a hole, said expandable member arranged in said interior, said expandable member absorbing said predetermined liquid that passes through said hole from the outside of said shell.

3. The switching mechanism as claimed in claim 2, wherein said switching mechanism comprises a partition member that separates a part of said interior from the other space in said interior,
said second contact arranged in said other space,
said expandable member arranged in said part of said interior, said part of said interior communicating with the outside of said shell through said hole,
at least a part of said partition member deforming or moving by the expansion of said expandable member, and thereby displacing said second contact.

4. The switching mechanism as claimed in claim 3, wherein said partition member comprises an elastic member that deforms by the expansion of said expandable member.

5. The switching mechanism as claimed in claim 4, wherein said partition member further comprises a side wall that is located on said inner surface of said shell and that surrounds said part of said interior, said elastic member covering said part of said interior,
said elastic member deforming by the expansion of said expandable member, and thereby displacing said second contact.

6. The switching mechanism as claimed in claim 3, wherein said second contact is located on said part of said partition member.

7. The switching mechanism as claimed in claim 1, wherein said shell comprises a hole, said switching mechanism comprising a first cover member that covers said hole on an inner side of said shell,
said expandable member located in a apace between said first cover member and said shell or an inner space of said hole.

8. The switching mechanism as claimed in claim 1, wherein said switching mechanism comprises a first cover member that seals an interior space of said shell from its surroundings,
said expandable member located on the outside of said first cover member,
said first and second contacts arranged in the sealed interior space,
at least a part of said first cover member deforming or moving by the expansion of said expandable member, and thereby displacing said second contact.

9. The switching mechanism as claimed in claim 1, wherein said shell comprises a hole, said predetermined liquid that is absorbed by said expandable member passing through said hole or entering said hole,
said switching mechanism comprising a second cover member capable of disintegrating by contact with a specific liquid, said second cover member covering said hole.

10. The switching mechanism as claimed in claim 9, wherein said expandable member absorbs said predetermined liquid after said second cover member disintegrates.

11. The switching mechanism as claimed in claim 9, wherein said second cover member is capable of disintegrating by contact with liquid with a pH within a predetermined range but not by contact with liquid with a pH beyond said predetermined range.

12. The switching mechanism as claimed in claim 9, wherein said cover member is dissolvable or decomposable by said specific liquid.

13. The switching mechanism as claimed in claim 1, wherein said predetermined liquid is a bodily fluid.

14. The switching mechanism as claimed in claim 1, wherein said device further comprises a second circuit unit electrically connectable to said first circuit unit, said first and second circuits unit electrically connecting with said first and second contacts, respectively,
the connection between said first and second circuit units being switched by electrically connecting or disconnecting said first contact to or from said first contact.

15. The switching mechanism as claimed in claim 14, wherein one of said first and second circuit units is a power-supply unit, and the other of said first and second circuit units is an operation circuit unit that is operated by electrical power supplied by said power-supply unit.
